(19) United States
(12) Patent Application Publication
(10) Pub. No.: US 2024/0173166 A1
(43) Pub. Date: May 30, 2024

(54) MASSAGE STONE HEATER

(71) Applicant: Jiangsu Mingteng Health Products Co., Ltd., Yancheng (CN)

(72) Inventor: Chuankui Du, Shanghai (CN)

(21) Appl. No.: 18/299,555

(22) Filed: Apr. 12, 2023

(30) Foreign Application Priority Data
Nov. 24, 2022 (CN) 202223134201.X

(51) Int. Cl. A61F 7/08; A61F 7/00; H05B 1/02

(52) U.S. Cl. CPC A61F 7/08; A61F 7/007; A61F 7/0085; H05B 1/0294; A61F 2007/0077; A61F 2007/0086; A61F 2007/0093; A61F 2007/0096

(57) ABSTRACT

The present disclosure relates to a massage stone heater, and belongs to the technical field of massage apparatuses. In the present disclosure, a user issues an instruction by means of operating the button of the controller, so that the circuit board in the controller supplies power to heating mechanisms inside the heating plates; the heating mechanisms generate heat after being powered on; and the heat of the heating mechanisms is conducted to the massage stones and the massage oil bottle through metal heat conduction plates of the heating plates.

### TECHNICAL FIELD

The present disclosure relates to the technical field of massage apparatuses, specifically to a massage stone heater.

### BACKGROUND

There are two types of existing massage stone heaters. One type is a heating oven. In use, water needs to be filled into the heating tank, and massage stones are submerged in the water. The heating oven heats the water to heat the massage stones to a preset temperature. The heating method using the heating oven is very complicated. For example, the water pre-filling process is time-consuming and troublesome; the heating speed is low; the process of raising the water temperature consumes additional electric energy; taking out the massage stones from the water easily wets operator's hands and easily scalds the arms; water needs to be disposed and heating tank needs to be cleaned after use. The other type is to use a heating bag to heat massage stones. An electric heating plate is arranged on one side of a heating bag. The massage stones are placed on the electric heating plate, and the electric heating plate generates heat to heat the massage stones. Although no water is used when using the heating bag, which saves the time for advance preparation, the heating plate on the single side can only heat a small number of massage stones, and the massage stones easily shake and collide with each other in a case. A high voltage of 110 V to 230 V easily causes electric shock and fire risk. Particularly for home massage service, electric shock and short circuit risks may be possibly caused once the heating bag gets wet by rainwater or once massage oil seeps and enters the electric heating plate.

### SUMMARY

An embodiment of the present disclosure aims to provide a massage stone heater, and aims at solving the problems of a heating oven caused by heating with water: complicated advance preparation, low heating efficiency, low heating speed, clients easily getting scalded, high power consumption, a waste of time and labor for cleaning after use, etc, and solving the problems caused by a single-sided heating structure of an existing heating bag: a small number of massage stones are heated; massage stones easily shake and collide with each other in the bag; a high voltage of 110 V to 230 V easily causes electric shock and short circuit, leading to a fire; etc. Therefore, a masseuse can heat more massage stones at one time; the massage stones can be managed according to different types; and a massage oil bottle can be heated at the same time.

In order to achieve the above purpose, the present disclosure provides the following technical solution:

A massage stone heater includes:
a case, which is divided into a left portion and a right portion, wherein lower ends of the left portion and the right portion are rotatably connected; the left portion and the right portion are rotatable by means of the connection of the lower ends; the left portion and the right portion are closed to form a complete case; hidden interlayers are respectively arranged on the left portion and the right portion;
heating plates, which include a left heating plate and a right heating plate respectively mounted in the hidden interlayers on the left and right sides of the case, wherein each heating plate is provided with a metal heat conduction plate towards an inner side of the case; each heating plate is provided with a thermal insulation layer towards an outer side of the case; a heating mechanism is mounted between each metal heat conduction plate and each thermal insulation layer; the metal heat conduction plates, the heating mechanisms and the thermal insulation layers are combined together;
heating mechanisms, which generate heat after being powered on;
temperature sensing devices, which are arranged on the left heating plate and the right heating plate and are used for sensing temperatures of the heating mechanisms; and
a controller, which is provided with a control mechanism and a circuit board.

As a further technical solution of the present disclosure, the circuit board is arranged inside the controller and electrically connected with the heating mechanisms inside the left heating plate and the right heating plate through lead wires, and supply power to the heating mechanisms inside the left heating plate and the right heating plate.

As a further technical solution of the present disclosure, the circuit board is arranged inside the controller and is electrically connected to and communicates with the temperature sensing devices of the left heating plate and the right heating plate respectively through lead wires; and the temperature sensing devices transmit sensed temperature signals to the circuit board in the controller through the lead wires.

As a further technical solution of the present disclosure, a working program is pre-stored in the circuit board; and the circuit board controls the heating mechanisms to perform heating and stop heating according to the pre-stored working program by means of powering on or powering off the heating mechanisms.

As a further technical solution of the present disclosure, the massage stone heater further includes a plurality of massage stone fixing devices respectively mounted on sides of the left portion and the right portion of the case close to the heating plates.

As a further technical solution of the present disclosure, the massage stone heater further includes at least one massage oil bottle heating fixing device mounted on one side of the left portion or the right portion of the case close to the heating plate.

As a further technical solution of the present disclosure, the massage oil bottle heating fixing device is applicable to a cylindrical or square massage oil bottle, and the massage oil bottle can be used for storing massage oil or lotion.

As a further technical solution of the present disclosure, the control mechanism of the controller is connected with the circuit board; and the control mechanism controls the circuit board and sends an instruction to the circuit board.

As a further technical solution of the present disclosure, the massage stone heater further comprises an external power supply; and the external power supply is electrically connected with the circuit board inside the controller and supplies power to the circuit board.

As a further technical solution of the present disclosure, a voltage supplied by the external power supply to the circuit board is a safe voltage less than 36 V.

Compared with the prior art, the present disclosure has the beneficial effects below. No water is used for heating; the heating speed is higher; the massage stones are easy to pick and place; the massage stone heater has low electric loss and is not required to be cleaned after use; a double number of massage stones can be heated at one time; the massage stones will not shake and collide with each other in the case; the safe voltage will not cause electric shock and lowers the risk of short circuit and fire; the massage stones are managed according to different types; and the massage stone heater can be used for heating massage oil.

### BRIEF DESCRIPTION OF THE DRAWINGS

FIG. 1 is a schematic product diagram of a massage stone heater;

FIG. 2 is a schematic product diagram 1 of a massage stone heater in an opened state;

FIG. 3 is a schematic product diagram 2 of a massage stone heater in an opened state;

FIG. 4 is a schematic exploded diagram of a massage stone heater; and

FIG. 5 is a schematic diagram of connection of electric heating components of a massage stone heater.

In the drawings: 1: case; 2: heating plate; 2-1: metal heat conduction plate; 2-2: heating mechanism; 2-3: thermal insulation layer; 3: temperature sensing device; 4: controller; 5: circuit board; 6: control mechanism; 7: massage stone fixing device; 8: massage oil bottle heating fixing device; 9: external power supply; and 10: zipper.

### DETAILED DESCRIPTION OF THE EMBODIMENTS

The technical solutions in the embodiments of the present disclosure will be described clearly and thoroughly below in combination with the accompanying drawings in the embodiments of the present disclosure.

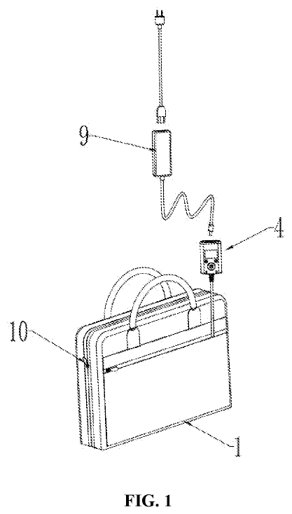

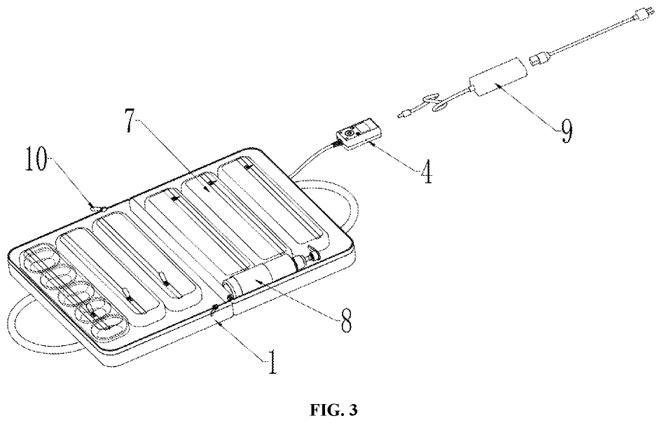

The embodiment of the present disclosure is achieved below. A massage stone heater as shown in FIG. 1 to FIG. 4 includes:
a case 1, metal heat conduction plates 2-1, heating mechanisms 2-2 and thermal insulation layers 2-3. The case 1 is made of polyester fabric. The case 1 is divided into a left portion and a right portion, and lower ends of the left portion and the right portion are connected by a canvas fabric. The left and right portions of the case 1 can rotate around the canvas fabric. The left and right portions are closed to form a complete case 1. The left and right portions of the case 1 can be connected by a zipper 10 after being closed. After the zipper 10 is zipped up, the case 1 is like a handbag. After the zipper 10 is unzipped, the case 1 can be opened in 180 degrees. The left portion and the right portion of the case 1 are respectively provided with hidden interlayers.

As shown in FIG. 1 to FIG. 4, the heating plates 2 include a left heating plate 2 and a right heating plate 2 respectively mounted in the hidden interlayers on the left and right sides of the case 1. Each heating plate 2 is provided with a metal heat conduction plate 2-1 towards an inner side of the case 1. Each heating plate 2 is provided with a thermal insulation layer 2-3 towards an outer side of the case 1. A heating mechanism 2-2 is mounted between each metal heat conduction plate 2-1 and each thermal insulation layer 2-3. The metal heat conduction plates 2-1, the heating mechanisms 2-2 and the thermal insulation layers 2-3 are combined together.

In the embodiment of the present disclosure, the heating mechanisms 2-2 are copper heating sheets, and the heating mechanisms 2-2 can generate heat after being powered on.

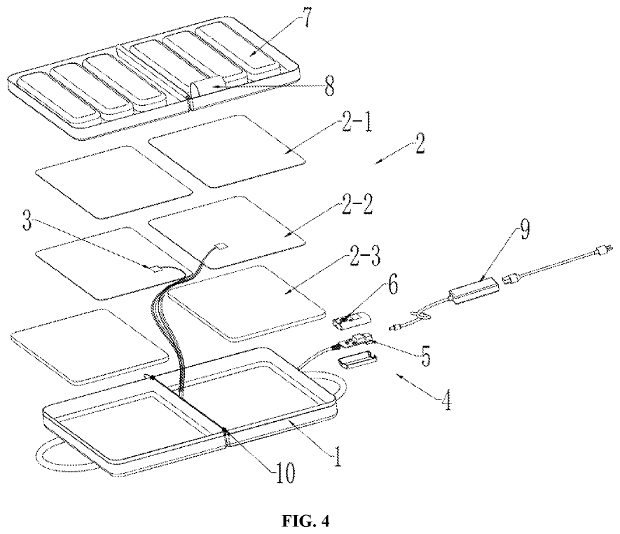

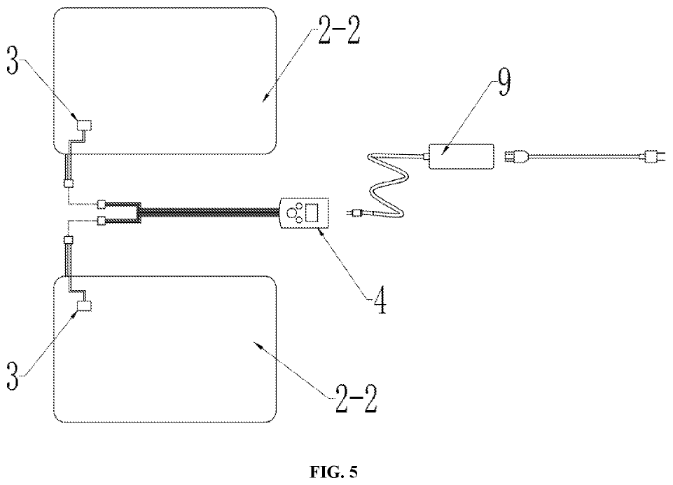

As shown in FIG. 4 to FIG. 5, temperature sensing devices 3 are respectively arranged on the heating mechanisms 2-2 on the left side and the right side and are used for sensing temperatures of the metal heat conduction plates 2-1.

As shown in FIG. 4, a circuit board 5 is mounted in the controller 4, and the controller 4 is provided with a control mechanism 6.

In the embodiment of the present disclosure, the control mechanism 6 is a button that is in contact with the circuit board 5. A working instruction can be sent to the circuit board 5 by means of the control mechanism 6.

As shown in FIG. 4 to FIG. 5, the circuit board 5 is arranged inside the controller 4. The circuit board 5 is electrically connected with the heating mechanisms 2-2 inside the left heating plate 2 and the right heating plate 2 through lead wires, and supply power to the heating mechanisms 2-2 inside the left heating plate 2 and the right heating plate 2.

As shown in FIG. 4 to FIG. 5, the circuit board 5 is arranged inside the controller 4. The circuit board 5 is electrically connected to and communicates with the temperature sensing devices 3 of the left heating plate 2 and the right heating plate 2 respectively through lead wires; and the temperature sensing devices 3 transmit sensed temperature signals to the circuit board 5 in the controller 4 through the lead wires.

In the embodiment of the present disclosure, a working program is pre-stored in the circuit board 5. The circuit board 5 controls the heating mechanisms 2-2 to perform heating and stop heating according to the pre-stored working program by means of powering on or powering off the heating mechanisms 2-2, so as to achieve the purpose of controlling a heating temperature.

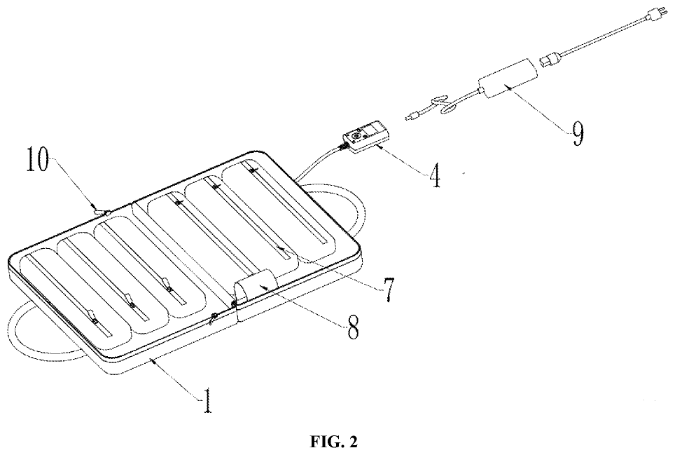

As shown in FIG. 2 to FIG. 4, the massage stone heater further includes a plurality of massage stone fixing devices 7 respectively mounted on sides of the left portion and the right portion of the case 1 close to the heating plates 2.

In the embodiment of the present disclosure, the massage stone fixing devices 7 adopts a plurality of cloth bags with zippers. Massage stones can be put into the cloth bags. After the zippers are zipped up, the massage stones can be prevented from moving and colliding with each other in the case 1.

As shown in FIG. 2 to FIG. 4, the massage stone heater further includes at least one massage oil bottle heating fixing device 8 mounted on at a position on one side of the case 1 close to the heating plate 2.

In the embodiment of the present disclosure, the massage oil bottle heating fixing device 8 is made of an elastic strap material which is sewed at the position on one side of the case 1 close to the heating plate 2, and is applicable to fixing a cylindrical or square massage oil bottle body. The massage oil bottle can be used for storing massage oil or lotion.

As shown in FIG. 4, the control mechanism 6 of the controller 4 is in contact with the circuit board 5. A user can control the circuit board 5 when the control mechanism 6 sends an instruction to the circuit board 5.

As shown in FIG. 1 to FIG. 5, the massage stone heater further includes an external power supply 9. The external power supply 9 is electrically connected with the circuit board 5 inside the controller 4 and supplies power to the circuit board 5.

In the embodiment of the present disclosure, the external power supply 9 is a power adapter. The external power supply 9 can be connected with an outer power supply, converts a high voltage of 110 V to 230 V to a safe voltage less than 36 V, and supplies the safe voltage to the circuit board 5.

In the embodiment of the present disclosure, the external power supply 9 is connected with the outer power supply to convert the high voltage of 110 V to 230 V to the safe voltage less than 36 V, and supplies the safe voltage to the circuit board 5. The user operates the control mechanism 6 of the controller 4 to instruct the circuit board 5 to start work, and sets an expected temperature value. After receiving the instruction of the control mechanism 6, the circuit board 5 supplies power to the heating mechanisms 2-2 inside the heating plates 2 on the left and right sides of the case 1 through lead wires according to the pre-stored program. The heating mechanisms 2-2 generate heat after being powered on, and transmit the heat to the metal heat conduction plates 2-1, and the metal heat conduction plates 2-1 transmit the heat to the massage stones in the massage stone fixing devices 7 on both sides of the case 1. The temperature sensing devices 3 mounted on the heating mechanisms 2-2 transmit sensed temperature values of the heating mechanisms 2-2 to the circuit board 5 inside the controller 4 by means of electronic information. When the temperature of the heating mechanism 2-2 on any one of the left side and the right side of the case 1 reaches or exceeds the expected temperature value set in the circuit board 5, the circuit board 5 stops supplying power to the heating mechanism 2-2 on this side according to the pre-stored program, and continues to supply power to the heating mechanism 2-2 on the other side until the temperature of this heating mechanism 2-2 also reaches or exceeds the expected temperature value set in the circuit board 5. When the temperature of the heating mechanism 2-2 on any one of the left side and the right side of the case 1 is less than the temperature preset in the circuit board 5, the circuit board 5 continues to supply power to the heating mechanism 2-2 on this side according to the pre-stored program.

In the embodiment of the present disclosure, a masseuse arranges the massage stones in the massage stone fixing devices 7 on the left and right sides of the case 1 in advance, then puts the massage oil bottle into the massage oil bottle heating fixing device 8, connects one end of the external power supply 9 to the outer power supply, connects the other end of the external power supply 9 to the controller 4, closes the case 1, and zips up the zipper 10 of the case 1. The masseuse instructs the massage stone heater to start heating through the control mechanism 6 of the controller 4.

In the embodiment of the present disclosure, the masseuse can instruct the massage stone heater to stop heating at any time through the control mechanism 6 of the controller 4.

For those skilled in the art, it is apparent that the present disclosure is not limited to the details of the demonstrative embodiments mentioned above, and that the present disclosure can be realized in other specific forms without departing from the spirit or basic features of the present disclosure. Therefore, from any point of view, the embodiments should be regarded as exemplary and non-limiting. The scope of the present disclosure is defined by the appended claims rather than the above description. Therefore, all changes falling within the meanings and scope of equivalent elements of the claims are intended to be included in the present disclosure. No drawing markings in claims shall be deemed to limit the claims involved.

In addition, it should be understood that although this specification is described in accordance with the implementation modes, and each implementation mode does not only contain one independent technical solution. This narration in the specification is only for clarity. Those skilled in the art should regard the specification as a whole. The technical solutions in all the embodiments can also be appropriately combined to form other implementation modes that can be understood by those skilled in the art.

What is claimed is:

1. A massage stone massager, comprising:
a case, which is divided into a left portion and a right portion, wherein lower ends of the left portion and the right portion are rotatably connected; the left portion and the right portion are rotatable by means of the connection of the lower ends; the left portion and the right portion are closed to form a complete case; hidden interlayers are respectively arranged on the left portion and the right portion;
heating plates, which include a left heating plate and a right heating plate respectively mounted in the hidden interlayers on the left and right sides of the case, wherein each heating plate is provided with a metal heat conduction plate towards an inner side of the case; each heating plate is provided with a thermal insulation layer towards an outer side of the case; a heating mechanism is mounted between each metal heat conduction plate and each thermal insulation layer; the metal heat conduction plates, the heating mechanisms and the thermal insulation layers are combined together;
heating mechanisms, which generate heat after being powered on;
temperature sensing devices, which are arranged on the left heating plate and the right heating plate and are used for sensing temperatures of the heating mechanisms; and
a controller, which is provided with a control mechanism and a circuit board.

2. The massage stone heater according to claim 1, wherein the circuit board is arranged inside the controller and electrically connected with the heating mechanisms inside the left heating plate and the right heating plate through lead wires, and supply power to the heating mechanisms inside the left heating plate and the right heating plate.

3. The massage stone heater according to claim 1, wherein the circuit board is arranged inside the controller and is electrically connected to and communicates with the temperature sensing devices of the left heating plate and the right heating plate respectively through lead wires; and the temperature sensing devices transmit sensed temperature signals to the circuit board in the controller through the lead wires.

4. The massage stone heater according to claim 2, wherein a working program is pre-stored in the circuit board; and the circuit board controls the heating mechanisms to perform heating and stop heating according to the pre-stored working program by means of powering on or powering off the heating mechanisms.

5. The massage stone heater according to claim 1, wherein the massage stone heater further comprises a plurality of massage stone fixing devices respectively mounted on sides of the left portion and the right portion of the case close to the heating plates.

6. The massage stone heater according to claim 1, wherein the massage stone heater further comprises at least one massage oil bottle heating fixing device mounted on one side of the left portion or the right portion of the case close to the heating plate.

7. The massage stone heater according to claim 6, wherein the massage oil bottle heating fixing device is applicable to a cylindrical or square massage oil bottle, and the massage oil bottle is used for storing massage oil or lotion.

8. The massage stone heater according to claim 1, wherein the control mechanism of the controller is connected with the circuit board; and the control mechanism controls the circuit board and sends an instruction to the circuit board.

9. The massage stone heater according to claim 1, wherein the massage stone heater further comprises an external power supply; and the external power supply is electrically connected with the circuit board inside the controller and supplies power to the circuit board.

10. The massage stone heater according to claim 9, wherein a voltage supplied by the external power supply to the circuit board is a safe voltage less than 36 V.
